(19) United States
(12) Patent Application Publication
(10) Pub. No.: US 2026/0083647 A1
(43) Pub. Date: Mar. 26, 2026

(54) TOPICAL DELIVERY SYSTEMS AND METHODS OF USE

(71) Applicant: Roger H. Tall, Idaho Falls, ID (US)

(72) Inventor: Roger H. Tall, Idaho Falls, ID (US)

(21) Appl. No.: 19/339,315

(22) Filed: Sep. 25, 2025

Related U.S. Application Data
(60) Provisional application No. 63/699,154, filed on Sep. 25, 2024.

(51) Int. Cl. A61K 8/06; A61Q 9/02

(52) U.S. Cl. CPC A61K 8/066; A61Q 9/02; A61K 2800/594; A61K 2800/596

(57) ABSTRACT

Topical delivery systems, multi-phase topical application formulations, and methods of use including combining multi-phase ingredients to form a first emulsified system, such as a pre-mixture hydrogel. A second emulsified system is formed to be combined with the pre-mixture hydrogel to form an emulsified topical application formulation. Additives and/or adjuvants are combined with the emulsified multi-phase formulation to form a delivery system. Delivery systems include a combined sustained-release, moisturizing barrier which may be applied to the skin. Multivesicular and/or multi-lamellar characteristics of the barrier provide for time-dependent effects, such as hair follicle softening and/or delayed active ingredient delivery.

### CROSS REFERENCE TO RELATED APPLICATIONS

This application claims the benefit and priority of U.S. Provisional Application Ser. No. 63/699,154, filed Sep. 25, 2024, all of which is hereby incorporated by reference herein in its entirety for all purposes.

### FIELD OF THE INVENTION

This invention generally relates to topical delivery systems and compositions, including pharmaceutical, cosmetic, and/or personal hygiene, topical application formulations and methods of use and is more specifically related to topical delivery compositions comprising colloids, emollients, emulsions, and hydrogels formulated for application to the skin of an animal or human for water retention, hydration, abrasion resistance, lubrication, and similar uses.

### BACKGROUND OF THE DISCLOSURE

In the field of pharmaceutical, cosmetic, personal hygiene products, topical application formulations and multi-phase applications, water is often necessary for application, product removal, and abrasion resistance.

In the field of shaving products, luxury shaves often only come with the luxury of time, which most do not have. However, a quick shave usually results in razor burn, scraped or injured skin, epidermis removal together with hair removal, and at the very least, dry, itchy, irritated skin. Skin bumps are also common when shaving, due to hardened hair follicles. Even the sharpest razor may pull on hardened follicles, raising them above the surface of the skin, then cutting the stretched, hardened follicles. Upon cutting, the hair shafts retract due to decreased tension, often burrowing into the dermis rather than maintaining a normal follicle path. The condition is often more pronounced with curly or less straight hair.

Although there have been great advances in the fields of shaving products and pharmaceutical, cosmetic, personal hygiene products, topical application formulations and multi-phase applications, considerable shortcomings remain.

### DESCRIPTION OF THE DRAWINGS

The accompanying drawings illustrate preferred embodiments of the invention, including iterations, permutations, or variations of the preferred embodiments. The accompanying drawings include information pertinent to the disclosure, in which:

FIGS. 1 and 1A show components of a topical delivery system, according to embodiments of the present disclosure.

FIG. 2 show a method of forming a hydrogel for a topical delivery system, according to embodiments of the present disclosure.

The features discussed and depicted in the accompanying drawings, as well as other features, are best understood from the detailed description of the preferred embodiments provided below, which is provided in connection and with reference to the accompanying drawings.

### DETAILED DESCRIPTION

The invention is described below in terms of exemplary and preferred embodiments. However, the invention is not limited to these embodiments. Rather, the present invention should be construed broadly to include other variants and embodiments of the invention that may be made by those skilled in the art without departing from the scope, range, and general spirit of the invention and its equivalents.

Emollients may be broadly defined as ingredients in skin care products to enhance hydration, soothe irritation or dryness, and prevent dehydration due to prolonged exposure to wind, heat, and sun. Emollients may also be recommended for conditions such as psoriasis, eczema, dryness, and/or scaling.

One category of emollients may include humectants, which attract water molecules, form bonds with those molecules, and retain the molecules close to the surface of the skin. Humectants may include multiple hydrophilic groups (e.g., hydroxyl groups), amines, and carboxyl groups, which may or may not undergo esterification.

Another category of emollients includes occlusives, which are immiscible in water. Occlusives generally do not attract water molecules but do help retain water molecules already present in the skin by forming a barrier of protection. Occlusives may include, but are not limited to, petroleum jelly, dimethicone, mineral oil, castor or jojoba oils, shea or cocoa butter, lanolin, waxes, liquid paraffin, and squalane.

Hydrogels are biocompatible, hydrated, multi-phase, polymeric materials (e.g., biphasic materials), where at least one phase, such as a water-immiscible polymer, is suspended by an interstitial fluid in another phase, which generally comprises water. Hydrogels may have a highly cross-linked, three-dimensional (3D) meshwork structure with elastic, swelling, or collapsing characteristics that may depend on the level of hydration of the structure. Hydrogels may be adapted for specific topical applications by modifying their cross-linking, chemical structure, morphology, or manufacturing process.

Colloids are two-phase systems that have one insoluble substance dispersed or suspended in another substance. Colloids may include emulsions.

Emulsions are broadly classified as mixtures of two or more liquids that are generally immiscible but undergo a mixing process that disperses one of the immiscible liquids (e.g., dispersed phase) into the other liquid (e.g., continuous phase). Emulsions may contain surfactants or emulsifiers that facilitate the interaction, dispersion, and stability of the two or more generally immiscible liquids.

Potato starch, as described herein, includes one or more starches from a Solanum tuberosum (e.g., Burbank russet potato), which contains about 20% starch by weight. The one or more starches include amylose and amylopectin. Amylopectin and amylose have a chemical formula including a repeating polymeric chain of [C₆H₁₀O₅]ₙ molecules. However, amylopectin differs from amylose in that amylopectin is a polysaccharide with a branched, complex structure including α(1→4) and α(1→6) glycosidic bonds; whereas amylose is a polysaccharide with a relatively more simple, linear structure including α-D-glucose units linked by α(1→4) glycosidic bonds.

One category of emulsions is oil in water (O/W) emulsions, where an oil is the dispersed phase and water and/or water-soluble ingredients comprise the continuous phase or the dispersing medium.

Another category of emulsions is water in oil (W/O) emulsions, where water or water-soluble ingredients comprise the dispersed phase and oil is the continuous phase or the dispersing medium.

As used herein, a vesicle refers to a component of the dispersed phase of an emulsion, or an O/W component of a two-phase emulsion system, many of which create a multivesicular emulsion. The vesicle may be shaped by the continuous phase, often exhibiting spherical, ovular (i.e., ovoid), or sometimes cylindrical shapes. The vesicle often comprises concentric shapes within the outer vesicle shell. When this occurs, each concentric shape has one or more inner O/W phases suspended in the outer, abutting external O/W phase. Although single micelles of O/W phase may comprise a vesicle, due to the type, contents, and mixture of the emulsion, single micelles are rare and the majority of vesicles within the multivesicular emulsion comprise the vesicles having iterative O/W layers or concentric shapes formed within the outer external O/W phase.

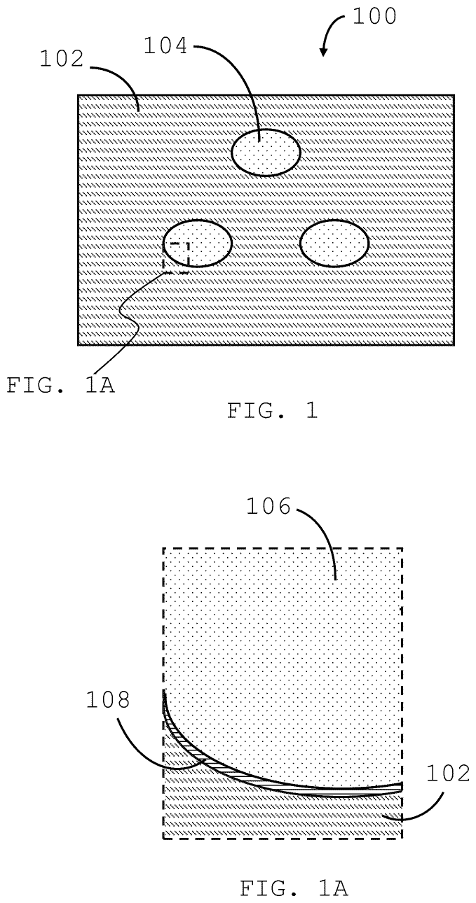

Referring now to FIGS. 1 and 1A, a topically delivery system 100 is disclosed. The topical delivery system 100 comprises a continuous phase 102 having one or more vesicles 104 dispersed throughout the continuous phase 102. The vesicle 104 is comprising a dispersed phase 106, which together with the continuous phase 102 form a barrier 108.

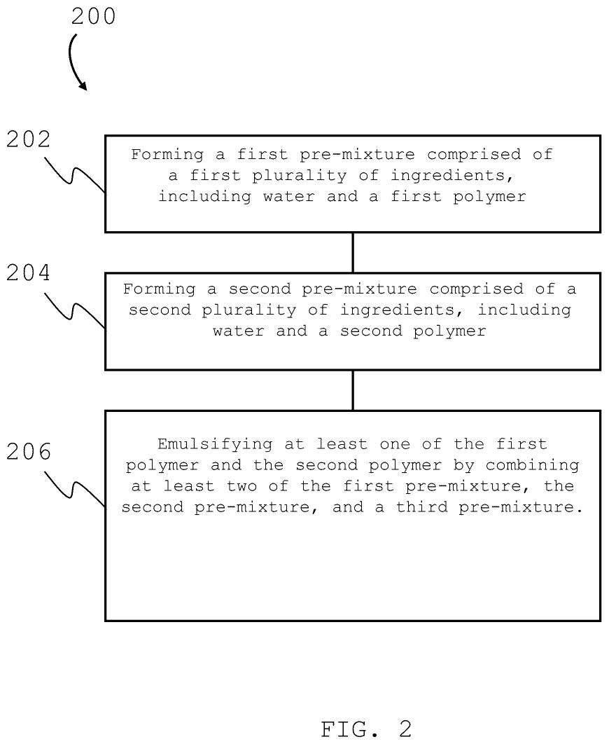

Referring now to FIG. 2, an embodiment of a method 200 of forming a hydrogel includes one or more steps 202, including forming a first pre-mixture, and one or more steps 204, including forming a second pre-mixture. The first pre-mixture is formed by combining multiple phases of ingredients. The first phase of ingredients includes, but is not limited to, distilled water and a compound produced from the hydrolysis of natural fats and/or oils (e.g., glycerin). A second phase includes a polymeric compound such as castor oil. The second phase further comprises cetearyl alcohol, isopropyl myristate, PEG-100 stearate (polyethylene glycol ester of stearic acid or polyglyceryl-2-stearate), glyceryl stearate, and stearyl alcohol. A third phase includes distilled water and a polymer chain of multiple glucose units (e.g., potato starch).

The second pre-mixture is formed by combining multiple phases of ingredients. The first phase of ingredients includes, but is not limited to, water and triethanolamine. A second phase of ingredients includes, but is not limited to, a co-solvent system comprising cyclohexane and ethyl acetate, which undergo a polymerization reaction to form a polyacrylate polymer (e.g., Carbomer 980).

A third pre-mixture may also be formed. The third pre-mixture is formed by combining the first pre-mixture and the second pre-mixture, thereby forming a hydrogel. The hydrogel may be a pre-mixture of a multi-phase formulation for a topical delivery system. The method 200 may further include one or more steps 206, including emulsifying at least one of a first polymer formed in the one or more steps 202 and a second polymer formed in the one or more steps 204 by combining at least two of the first pre-mixture, the second pre-mixture, and the third pre-mixture.

Another embodiment of a method of forming a hydrogel includes forming a first pre-mixture and a second pre-mixture. In this embodiment, the first pre-mixture is also formed by combining multiple phases of ingredients. The first phase of ingredients includes, but is not limited to, distilled water and a modified potato starch. The modified potato starch is formed by presenting a potato slurry to a form of electromagnetic radiation having frequencies ranging from 300 MHz to 300 GHz (e.g., microwaves) for about one minute. It is noted that modified starch may increase the pronounced effect, or release, of amylopectin and the amount of water in a starch gel that is formed. In some embodiments, the modified potato starch may be further modified by the addition of an alcohol preservative.

The second pre-mixture is formed by combining multiple phases of ingredients. The first phase of ingredients includes, but is not limited to, pH-adjusted, agitated water, where triethanolamine was used to adjust the pH and the water is being agitated at a moderate mixing level. A second phase of ingredients includes, but is not limited to, a polymerized solute (e.g., Carbomer 980, or a cross-linked polyacrylate polymer with one or more molecular structures of C₃H₄O₂, such as a repeating [C₃H₄O₂]ₙ molecular unit) that is slowly added to the agitated water.

In this embodiment, a third pre-mixture may also be formed. The third pre-mixture is a stable-sustained-release (SSR) hydrogel. The SSR hydrogel is formed by combining the first pre-mixture and the second pre-mixture using intermittent immersion blending or mixing with a high-shear mixer (e.g., Silverson Mixer) until the second pre-mixture is uniformly dispersed within the first pre-mixture. The SSR hydrogel may be stored at ambient room temperatures without substantial structural alterations for extended periods of time (e.g., greater than or equal to one week).

In some embodiments, a multi-phase formulation for a topical delivery system is formed by adding a biodegradable fluid, such as ethyl propylene glycol acetal levulinate (e.g., Ecosmooth™ 1100, or 1,3-Dioxolane-2-propanoic acid, 2,4-dimethyl-, ethyl ester), to a hydrogel that is at a temperature of less than or equal to 45° C., and mixing until the biodegradable fluid is dispersed within the hydrogel. Additional low speed, inert mixing (e.g., such as with a silicone spatula) in a cooled (e.g., refrigerated) environment may accelerate the emulsification process. In other embodiments, the multi-phase formulation may be formed by adding one or more silicone-based emulsifiers to the hydrogel, including but not limited to, sorbitan oleate, PEG-30 dipolyhydroxystearate, polysorbate 60, and cyclopentasiloxane (e.g., having a general chemical formula comprising C₁₀H₃₀O₅Si₅).

In some embodiments, a topical delivery system is formed by combining one or more additives and/or adjuvants with an emulsified multi-phase formulation. Additives and adjuvants may include, but are not limited to, vitamins, emollients, fragrances, and “green surfactants”. For example, additives and/or adjuvants may include, but are not limited to, vitamin A, vitamin E, and/or vitamin C; preservatives such as one or more parabens, DMDM hydantoin, 1,2 octanediol, and/or organic acids; sunscreens such as zinc oxide, titanium dioxide, avobenzone, octinoxate, and/or oxybenzone; hyaluronic acids; anti-acne agent (e.g., salicylic acid); antibiotic; contraceptive; hemorrhoid treatment; vaginal treatment; antiperspirants; insect repellant; and natural or synthetic essential oils. The percentage of the additives and/or adjuvants may be from 0.001 to 0.05 (% w/w). For instance, 1000 grams of a topical delivery system with additives and/or adjuvants may comprise about 700 grams water, 100 grams glycerin, 40 grams cetearyl, 40 grams castor oil (e.g., comprising a general chemical formula of C₅₇H₁₀₄O₉ although other forms are contemplated and included herein), 40 grams myristate, 40 grams GS+PEG-100, 15 grams of modified potato starch, 20 grams Carbomer 980, and 5 grams of additives and/or adjuvants.

Generally, the continuous phase comprises one or more OH molecules. For example, the continuous phase comprises a molecule of the general formulas:

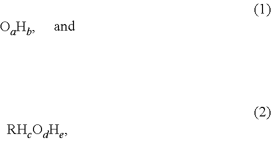

wherein Formula (1), a and bare generally any of 1 and 2, such as with water and hydroxyl molecules, and in Formula (2), R is C_{f}, where f is any number from 0, 1, to 57; c, d, and e, are any number from 0, 1, to 104, such as in cetearyl alcohol (e.g., c=72, d=2, and f=34), stearyl alcohol, (c=38, d=1, and f=18), sugar alcohols (e.g., where a repeating pattern, n, is formed having c=1, d=1, and f=1, and outside the repeating pattern, e=2), and other OH molecules.

Generally, the dispersed phase comprises a general formula for a molecular unit of a monomeric or polymeric compound:

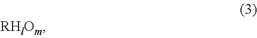

wherein Formula (3), R comprises one or more of an amine group, a silicone ion or cation, and Cₖ, where k is any number 3 to 57, and/is any number 8 to 104, and m is any number 1 to 14, such as ricinoleic acid (k=57, l=34, and m=3), which is a key ingredient in castor oil, lauric acid (k=12, l=24, and m=2), which is a key ingredient in coconut oil, glycerin (k=3, l=8, m=3), isopropyl myristate (k=17, l=34, and m=2), palmitic acid (k=16, l=32, m=2), oleic acid (k=18, l=34, and m=2), which is a key ingredient in shea butter, and ethylhexyl palmitate (k=24, l=48, and m=2).

In some embodiments, the formulation comprises a continuous phase comprising, for example, water or distilled water. The continuous phase may further comprise one or more solvents, such as ethanol, propylene glycol, glycerin, stearyl alcohol, isopropyl alcohol, cetearyl alcohol, and combinations thereof.

In some embodiments, the formulation comprises a dispersed phase comprising, for example, glycerin, natural oils (e.g., castor oil, coconut oil, cocoa oil, palm oil, etc.), and combinations thereof.

In some embodiments, surfactants may be formed by using a carboxyl or hydroxyl group to react with a carboxylic acid. For example, a surfactant may be formed by an esterification of the amino acid (e.g., leucine) with an alcohol, reductive amination, and methylation of a secondary amine. Esterification may occur in some instances using hydrogen peroxide in a Baeyer-Villiger oxidation reaction, although other means of esterification are contemplated herein.

In some embodiments, a surfactant may be formed by acid hydrolysis of dimethyldichlorosilane to form a hydrolysate mixture of linear and cyclic polysiloxanes (e.g., dimethylsiloxanes and polydimethysiloxanes) from which cyclic siloxanes can be removed by distillation in the presence of a base (e.g., potassium hydroxide). In some embodiments, the surfactant is obtained from a distributor, such as Dow Chemical, Clariant, Alfa Chemistry, Syensqo, Mckinley Resources, or Mison Commercial. The surfactant may comprise coco-betaine, polysorbate, oleyl betaine, cocamidopropyl betaine, phospholipid PTC, glycerin, and combinations thereof.

The surfactant may be combined with a viscosity-adjusting emulsifier, such as a fatty alcohol emulsifier. In some embodiments, the viscosity-adjusting emulsifier comprises one or more of cetearyl or cetearyl alcohol, coconut oil, vitamin E, aloe vera, almond oil, sunflower oil, avocado oil, olive oil, a mixture of cetyl alcohol and stearic acid, lecithin, and combinations thereof. The ratio of the amount of viscosity-adjusting emulsifier to the amount of surfactant may be from 2:1, 3:1, 4:1, to 5:1.

In some embodiments, a polymer is added to the surfactant system. For example, EcoSmooth™ (Dow Chemical, having a general chemical formula of C₁₀H₁₈O₄ or C₁₀H₁₈O₅), silica, one or more starches (e.g., potato starch, corn starch, etc.), a carbomer (e.g., Carbomer 940, Carbomer 980, etc.), PEG-100, and/or other natural or synthetic polymers may be added.

Additional viscosity-adjusting additives may be used to optimize the viscosity of the formulation for the intended use. For example, fructan gum, xanthan gum, sclerotium gum, Carbomer (e.g., Carbomer 940, Carbomer 980, etc.), beeswax, potato starch, corn starch, silica, acrylates, polyacrylic acid thickeners (e.g., Covacryl), polyethylene glycol (e.g., PEG-100 stearate), and combinations thereof.

Additives may include moisturizers, such as lanolin alcohol, glycerin, butylene glycol, propylene glycol, myristate (e.g., isopropyl myristate), salts of myristic acid (e.g., aluminum isostearates/myristate, aluminum myristate, calcium myristate, potassium myristate, zinc myristate, sodium myristate, etc., lanolin derivatives, dimethicone, phenyl trimethicone, cyclomethicone, and combinations thereof.

Additional additives may include, but are not limited to, buffering agents, pH adjusters, abrasives, anticaking agents (e.g., sodium aluminosilicate, calcium silicate, powdered cellulose, magnesium stearate, sodium bicarbonate, etc.), absorbents, preservatives (e.g., sorbic acid, sodium benzoate, methylparaben, etc.), fragrances, colors, and combinations thereof.

In some embodiments, topical delivery systems discussed herein have one or more emollients with time-dependent effects and characteristics, such as time-released hydration to skin surfaces, softening of hair follicles prior to shaving, time-released vitamin delivery to the skin, and combinations thereof. Other time-dependent and time-independent effects and improvements may include skin rehydration, moisturizing, preventing or delaying dehydration at points of application, lubrication, abrasive resistance or utilizing a combined sustained-release, moisturizing barrier (e.g., such as barrier 108) to prevent dryness, irritation, and skin “bumps”. In some embodiments, the combined sustained-release, moisturizing barrier 108 may be formed by shear mixing oil in water or water in oil with ingredients specifically tailored to one or more of the desired time-dependent effects. Specifically tailored ingredients of the barrier 108 include at least one of a modified potato starch and an ester (e.g., Ecosmooth™). Multivesicular and/or multi-lamellar characteristics of the barrier 108 provide for time-dependent effects, such as hair follicle softening and/or delayed active ingredient delivery.

In some embodiments, the topical delivery system and/or multi-phase topical application comprises a multivesicular emulsion formed from a quaternary ammonium salt, such as behentrimonium methosulfate, behenamidopropyl ethyldimonium ethosulfate, benzalkonium chloride (BAC), or amino-acid-based “green surfactants”, such as lysine- or histidine-based single chain or dimeric surfactants. In other embodiments, the quaternary amino-acid based surfactants comprise leucine- and methionine-based surfactants. The chain lengths of some of the quaternary ammonium compounds used herein may be from C10, C12, and C14 lengths.

Example embodiments of a shaving application formulation may comprise about 12-18% Ecosmooth™ dispersed in an emulsion comprising about 40-70% water, 7-11% glycerin, 2-4% cetearyl, 2-4% castor oil, 2-4% myristate, 2-4% GS+PEG-100, 0.5-1.5% modified potato starch, 1.0-2.0% Carbomer 980, and 0.001-0.05% additives and/or adjuvants, where percentages are by weight (e.g., % w/w) and where at least the cetearyl, castor oil, myristate, and GS+PEG-100 are dispersed (e.g., comprise the dispersion phase) in the water and glycerin (e.g., comprise the continuous phase). For example, 1000 grams of the shaving application formulation may comprise about 600 grams water, 90 grams glycerin, 30 grams cetearyl alcohol, 30 grams castor oil, 30 grams isopropyl myristate, 30 grams GS+PEG-100, 150 grams Ecosmooth™, 15 grams of modified potato starch, 20 grams Carbomer 980, and 5 grams of additives and/or adjuvants.

Additional methods of use may include applying one or more topical application formulations described herein to the surface of the skin of an animal or human and shaving the hair of the skin beneath the topical application. In some embodiments, a period exists between the application and the shaving, such that softening occurs in the meantime. In other embodiments, positive effects of a combined sustained-release, moisturizing barrier are obtained by allowing a film to form after application and/or shaving. For example, many shaving products instruct users to rinse skin after shaving. In embodiments described herein, the combined sustained-release, moisturizing barrier is allowed to form when rinsing does not occur after shaving (i.e., residue is left on the skin after application and/or shaving).

Examples of formulations, methods of manufacture, and product formulations of the present invention are set forth below.

### Example 1

Ingredients used in this, Example 1, comprise the following:

TABLE 1
Ingredients to be used for different phases in formulating
a hydrogel, an emulsion, and/or a skin care product

| Phase A | Phase B | Phase C | Phase D | Phase E |
|---|---|---|---|---|
| Ingredient 1: Distilled | Ingredient 1: Castor | Ingredient 1: Distilled | Ingredient 1: Distilled | Ingredient 1: |
| Water | Oil (Ricinus | Water | Water | Ecosmooth |
| | Communis Seed Oil) | | | 1100 (ethyl PG- |
| | | | | acetal |
| | | | | levulinate) |
| Ingredient 2: | Ingredient 2: Cetaryl | Ingredient 2: Potato | Ingredient 2: | |
| Glycerin (Glycerine) | Alcohol | Starch (solanum | Carbomer 980 | |
| | (distearoylethyl | tuberosum starch) | (polyglyceryl-2- | |
| | dimonium chloride) | | stearate (and) | |
| | | | glyceryl stearate | |
| | | | (and) stearyl alcohol) | |
| | Ingredient 3: | | Ingredient 3: | |
| | Isopropyl Myristate | | Triethanolamine-99 | |
| | | | (2,2′,2″- | |
| | | | nitrilotriethanol) | |
| | Ingredient 4: | | | |
| | GS + PEG-100 | | | |
| | (polyglyceryl-2- | | | |
| | stearate (and) | | | |
| | glyceryl stearate | | | |
| | (and) stearyl alcohol | | | |

In this, Example 1, a method of formulating a first phase (e.g., Phase A) of ingredients includes: charging a first beaker with the Phase A ingredients, heating the Phase A ingredients of Table 1 to about 60° C. to 70° C., and mixing with moderate propeller speed, such as 500 rpm to 1000 rpm, thereby forming a heated water phase (e.g., continuous phase).

A method of formulating a second phase (e.g., Phase B) of ingredients includes: charging a second beaker with each of the Phase B ingredients, heating the Phase B ingredients of Table 1 to about 60° C. to 70° C., mixing with moderate propeller speed, such as about 400 rpm, 500 rpm, to about 1000 rpm, and allowing a first transformational change (e.g., melting) to occur within the mixture, thereby forming a heated oil phase (e.g., dispersed phase).

The transformed Phase B ingredients are combined with the heated/mixed Phase A ingredients to form an emulsion. For example, mixing to the combination may occur using a high propeller speed, such as greater than 1000 rpm. In this, Example 1, a high-speed immersion blender set to high is used for about eight (8) to ten (10) minutes of emulsifying mixing. Then, for about four (4) to five (5) minutes, the immersion blender is set to low for intermittent mixing prior to cooling.

A method of formulating a third phase (e.g., Phase C) of ingredients includes: pre-mixing a potato starch slurry using the water and starch listed in the Phase C ingredients of Table 1, heating the slurry to about 60°, 65°, to 70° C., mixing with low propeller speed (e.g., 50 rpm, 80 rpm, 100 rpm, or 200 rpm), transforming/modifying the mixed/heated ingredients by, for example, microwaving for about one 50 seconds to (1) minute. It is noted that modified starch increases the release of amylopectin and the amount of water in a starch gel that is formed.

Combine the transformed Phase C ingredients with the emulsified Phase B ingredients. Slowly mix this combination using moderate propeller speed (e.g., 400 rpm, 500 rpm, to 1000 rpm) until the Phase C ingredients are dispersed in the emulsified ingredients to form a humectant-modified emulsion.

A method of formulating a fourth phase (e.g., Phase D) of ingredients includes: adding the water of the Phase D ingredients of Table 1 to a separate container, adjusting the pH of the water to about seven (7) with drops of the triethanolamine-99 of the Phase D ingredients (if necessary), and controlled addition of small amounts of Carbomer 980 (e.g., 1,3-Propanediol, 2-(chloromethyl)-2-((2-naphthyloxy)methyl)-) to the surface of the water while mixing at moderate propeller speed (e.g., 400 rpm to 1000 rpm).

Combine the mixed Phase D ingredients with the humectant-modified emulsion using intermittent immersion blending (e.g., with a Silverson blender/mixer) until the Phase D ingredients are dispersed within the humectant-modified emulsion ingredients to form a hydrogel formulation or a hydrogel pre-mixture. A hydrogel formulation or pre-mixture may be stored between about 10°, 15°, 20°, 25°, to about 30° C., until it is added to a Phase A/Phase B mixture of ingredients.

A method of formulating a fifth phase (e.g., Phase E) of ingredients includes: cooling a hydrogel formulation or a hydrogel pre-mixture to about 40°, 45°, to about 50° C., or cooler, adding the Phase E ingredient(s) of Table 1 (e.g., Ecosmooth 1100), mixing with an immersion blender on moderate to low speeds until the Phase E ingredient(s) is dispersed within the hydrogel, cooling in a moisture-controlled environment (e.g., covered container) at ambient temperature, and slightly mixing at very low speeds (e.g., 50 rpm to 60 rpm or lower, such as with hand-mixing).

Amounts of different phases of ingredients to be used in this, Example 1, are given below in Table 2, which references the ingredients listed in Table 1.

TABLE 2
Amounts of ingredients of different phases (by % w/w) relative to Table 1

| Phase A | Phase B | Phase C | Phase D | Phase E |
|---|---|---|---|---|
| Ingredient 1: 30-60% | Ingredient 1: 2-5% | Ingredient 1: 8-12% | Ingredient 1: 8-12% | Ingredient 1: 10-20% |
| Ingredient 2: 7-12% | Ingredient 2: 2-5% | Ingredient 2: 1-3% | Ingredient 2: 0.5-1.5% | |
| | Ingredient 3: 2-5% | | Ingredient 3: Drops | |
| | | | (e.g., as with | |
| | | | titration-such as | |
| | | | about 0.05 mL) | |
| | Ingredient 4: 2-5% | | | |

### Example 2

The methods and formulations of this, Example 2, are similar if not identical to those of Example 1, except that the methods and formulations differ in that Ingredient 1 of Phase B of Table 1 comprises coconut oil. Additionally, the method of formulation of Phase E ingredients includes cooling the hand-mixed combination to about 0° to about 5° C. (as with refrigeration).

### Example 3

The methods and formulations of this, Example 3, are similar if not identical to those of Example 1, except that the formulations differ according to ingredients and amounts listed in Table 3 and Table 4 (below). Additionally, the methods and formulations of this, Example 3, differ from those of Example 1 in that the Phase C ingredients may be modified by adding an occlusive, such as Dowsil™ 2502 (i.e., microwaving is omitted from the methods of this example), formulating Phase E ingredients includes cooling the hand-mixed combination to about 0° to about 5° C. (as with refrigeration), and formulating the Phase E ingredients may include adding one or more natural or synthetic fragrances (e.g., cyclic aromatic compounds) after cooling.

Ingredients used in this, Example 3, comprise the following:

TABLE 3
Ingredients to be used for different phases in formulating
a hydrogel, an emulsion, and/or a skin care product

| Phase A | Phase B | Phase C | Phase D | Phase E |
|---|---|---|---|---|
| Ingredient 1: Distilled | Ingredient 1: Castor | Ingredient 1: Distilled | Ingredient 1: Distilled | Ingredient 1: |
| Water | Oil (Ricinus | Water | Water | Ecosmooth 1100 |
| | Communis Seed Oil) | | | (ethyl PG-acetal |
| | | | | levulinate) |
| Ingredient 2: | Ingredient 2: Cetaryl | Ingredient 2: Potato | Ingredient 2: | Ingredient 2: |
| Glycerin (Glycerine) | Alcohol | Starch (solanum | Carbomer 980 | One or more |
| | (distearoylethyl | tuberosum starch) | (polyglyceryl-2- | natural or |
| | dimonium chloride) | | stearate (and) | synthetic oils |
| | | | glyceryl stearate | (aromatic |
| | | | (and) stearyl alcohol) | compounds) |
| | Ingredient 3: | Ingredient 3: | Ingredient 3: | |
| | Isopropyl Myristate | Dowsil ™ 2502 (cetyl | Triethanolamine-99 | |
| | | dimethicone) | (2,2′,2″- | |
| | | | nitrilotriethanol) | |
| | Ingredient 4: | | | |
| | GS + PEG-100 | | | |
| | (polyglyceryl-2- | | | |
| | stearate (and) | | | |
| | glyceryl stearate | | | |
| | (and) stearyl alcohol | | | |

Amounts of different phases of ingredients to be used in this, Example 3, are given below in Table 4, which references the ingredients listed in Table 3.

TABLE 4
Amounts of ingredients of different phases (by % w/w) relative to Table 3

| Phase A | Phase B | Phase C | Phase D | Phase E |
|---|---|---|---|---|
| Ingredient 1: 40-60% | Ingredient 1: 2-5% | Ingredient 1: 8-12% | Ingredient 1: 8-12% | Ingredient 1: 10-20% |
| Ingredient 2: 7-12% | Ingredient 2: 2-5% | Ingredient 2: 1-3% | Ingredient 2: 0.5-1.5% | Ingredient 2: 0.1-0.5% |
| | Ingredient 3: 2-5% | | Ingredient 3: Drops | |
| | | | (e.g., as with | |
| | | | titration-such as | |
| | | | about 0.05 mL) | |
| | Ingredient 4: 2-5% | | | |

### Example 4

The methods and formulations of this, Example 4, are similar if not identical to those of Example 1, except that the formulations differ according to ingredients and amounts listed in Table 5 and Table 6 (below). Additionally, the methods and formulations of this, Example 4, differ from those of Example 1 in that formulating Phase E ingredients includes cooling the hand-mixed combination to about 0° to about 5° C. (as with refrigeration), and formulating the Phase E ingredients may include adding one or more natural or synthetic fragrances (e.g., cyclic aromatic compounds) after cooling.

Ingredients used in this, Example 4, comprise the following:

TABLE 5
Ingredients to be used for different phases in formulating
a hydrogel, an emulsion, and/or a skin care product

| Phase A | Phase B | Phase C | Phase D | Phase E |
|---|---|---|---|---|
| Ingredient 1: | Ingredient 1: Castor | Ingredient 1: | Ingredient 1: | Ingredient 1: |
| Distilled Water | Oil (Ricinus | Distilled Water | Distilled Water | Xiameter ™ PMX- |
| | Communis Seed | | | 0245 |
| | Oil) | | | (Cyclopentasiloxane) |
| Ingredient 2: | Ingredient 2: Cetaryl | Ingredient 2: Potato | Ingredient 2: | Ingredient 2: |
| Glycerin (Glycerine) | Alcohol | Starch (solanum | Carbomer 980 | One or more natural |
| | (distearoylethyl | tuberosum starch) | (polyglyceryl-2- | or synthetic oils |
| | dimonium chloride) | | stearate (and) | (aromatic |
| | | | glyceryl stearate | compounds) |
| | | | (and) stearyl | |
| | | | alcohol) | |
| | Ingredient 3: | | Ingredient 3: | |
| | Isopropyl Myristate | | Triethanolamine-99 | |
| | | | (2,2′,2″- | |
| | | | nitrilotriethanol) | |
| | Ingredient 4: | | | |
| | GS + PEG-100 | | | |
| | (polyglyceryl-2- | | | |
| | stearate (and) | | | |
| | glyceryl stearate | | | |
| | (and) stearyl alcohol | | | |

Amounts of different phases of ingredients to be used in this, Example 4, are given below in Table 6, which references the ingredients listed in Table 5.

TABLE 6
Amounts of ingredients of different phases (by % w/w) relative to Table 5

| Phase A | Phase B | Phase C | Phase D | Phase E |
|---|---|---|---|---|
| Ingredient 1: 35-40% | Ingredient 1: 3.1-3.5% | Ingredient 1: 9-10% | Ingredient 1: 9-10% | Ingredient 1: 10-15% |
| Ingredient 2: 7-9% | Ingredient 2: 3.1-3.5% | Ingredient 2: 1.50-1.90% | Ingredient 2: 0.75-1.25% | Ingredient 2: 0.1-0.5% |
| | Ingredient 3: 3.1-3.5% | | Ingredient 3: Drops | |
| | | | (e.g., as with | |
| | | | titration-such as | |
| | | | about 0.05 mL) | |
| | Ingredient 4: 3.1-3.5% | | | |

While specific embodiments and applications of the present technology have been illustrated and described, it is to be understood that the technology is not limited to the precise configuration and components disclosed herein. Various modifications, changes, and variations which will be apparent to those skilled in the art may be made in the arrangement, operation, and details of the methods and systems of the present technology disclosed herein without departing from the spirit and scope of the technology.

What is claimed is:

1. A multi-phase hydrogel, comprising:
a first pre-mixture comprising a first plurality of ingredients, including water and a first polymer;
a second pre-mixture comprising a second plurality of ingredients, including water and a second polymer;
wherein at least one of the first polymer and the second polymer comprises modified potato starch.

2. The multiphase hydrogel of claim 1, further comprising:
a third pre-mixture comprising a combination of the first pre-mixture and the second pre-mixture.

3. The multiphase hydrogel of claim 1, wherein the first plurality of ingredients comprises castor oil.

4. The multiphase hydrogel of claim 1, wherein the first polymer comprises the modified potato starch including amylopectin, and the second polymer comprises a polyacrylate polymer.

5. A multiphase hydrogel formulation, comprising:
a continuous phase having a first molecule of at least one of the general formulas: 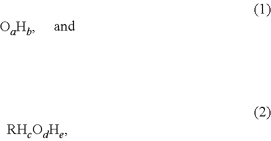
wherein Formula (1) a and b are any of 1 and 2;
wherein Formula (2), R is Cr, where f is any number from 1 to 57;
wherein c, d, and e, are any number 0, 1, to 104; and
a dispersed phase having at least a second molecule and a third molecule, the first molecule comprising a general formula: 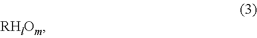
wherein Formula (3), R is Cₖ, k is any number 3 to 57, l is any number 3 to 104, and m is any number 1 to 14; and
wherein the third molecule has the Formula (3) where k is 6, l is 10, and m is 5 of a repeating polymeric chain.

6. The multiphase hydrogel formulation of claim 5, wherein the continuous phase further comprises a fourth molecule of the Formula (2), wherein a is 1, b is 2, c is 5, d is 3, and e is 3.

7. The multiphase hydrogel formulation of claim 5, further comprising a second continuous phase and a second dispersed phase,
wherein a is 1 and b is 2 in the first continuous phase and the second continuous phase;
wherein k is 57, l is 104, and m is 9 in the first dispersed phase; and
wherein k is 6, l is 10, and m is 5 in the second dispersed phase.

8. The multiphase hydrogel formulation of claim 5, further comprising a second dispersed phase, wherein k is 3, l is 4, and m is 2.

9. The multiphase hydrogel formulation of claim 8, further comprising a third dispersed phase, wherein k is 10, l is 18, and m is 5.

10. A method of forming a hydrogel, comprising:
forming a first pre-mixture comprising a first plurality of ingredients, including water and a first polymer;
forming a second pre-mixture comprising a second plurality of ingredients, including water and a second polymer;
emulsifying at least one of the first polymer and the second polymer by combining at least two of the first pre-mixture, the second pre-mixture, and a third pre-mixture, wherein the at least two of the first pre-mixture, the second pre-mixture, and a third pre-mixture comprises an esterified molecule.

11. The method of claim 10, wherein the emulsifying comprises combining the first pre-mixture and the second pre-mixture after storage of the third pre-mixture.

12. The method of claim 10, further comprising:
forming the third pre-mixture comprising a third plurality of ingredients, including water and a third polymer, wherein forming the third pre-mixture comprising a third plurality of ingredients, including water and a third polymer comprises:
adjusting a pH of the water of the third plurality of ingredients; and
agitating the water of the third plurality of ingredients.

13. The method of claim 10, wherein forming a second pre-mixture comprising a second plurality of ingredients, including water and a second polymer, comprises:
modifying the second polymer by presenting the second polymer to electromagnetic radiation.
